(19) United States
(12) Patent Application Publication
(10) Pub. No.: US 2025/0344732 A1
(43) Pub. Date: Nov. 13, 2025

(54) METHODS FOR CONTINUOUS GAS-LIQUID COUNTERCURRENT PREPARING SUGAR ALCOHOLS

(71) Applicants: INSTITUTE OF ZHEJIANG UNIVERSITY-QUZHOU, Quzhou (CN); ZHEJIANG HUAKANG PHARMACEUTICAL CO., LTD., Quzhou (CN)

(72) Inventors: Jie FU, Quzhou (CN); Haoan FAN, Quzhou (CN); Bolong LI, Quzhou (CN); Jianghao WANG, Quzhou (CN); Xiuyang LYU, Quzhou (CN); Mian LI, Quzhou (CN); Wulong YANG, Quzhou (CN); Yanlong AN, Quzhou (CN); Chengjun LIAO, Quzhou (CN)

(73) Assignees: INSTITUTE OF ZHEJIANG UNIVERSITY-QUZHOU, Quzhou (CN); ZHEJIANG HUAKANG PHARMACEUTICAL CO., LTD., Quzhou (CN)

(21) Appl. No.: 19/280,275

(22) Filed: Jul. 25, 2025

Related U.S. Application Data
(63) Continuation-in-part of application No. PCT/CN2024/125952, filed on Oct. 19, 2024.

(30) Foreign Application Priority Data
Feb. 27, 2024 (CN) 202410214665.6

(51) Int. Cl. A23L 27/30

(52) U.S. Cl. CPC A23L 27/34

(57) ABSTRACT

Provided is a method for continuous gas-liquid countercurrent preparing sugar alcohol, the method includes: through a gas-liquid countercurrent trickle bed reactor, pouring a biomass sugar solution from a liquid phase inlet into the trickle bed reactor from top to bottom, and introducing hydrogen gas from a gas phase inlet to the trickle bed reactor from bottom to top; the biomass sugar solution and the hydrogen gas pass through a catalyst bed in a gas-liquid countercurrent manner for hydrogenation reduction to obtain a biomass sugar alcohol solution.

### CROSS-REFERENCE TO RELATED APPLICATIONS

This application is a Continuation-in-part of International Application No. PCT/CN2024/125952, filed on Oct. 19, 2024, which claims priority to Chinese Patent Application No. 202410214665.6, filed on Feb. 27, 2024, the entire contents of each of which are hereby incorporated by reference.

### TECHNICAL FIELD

The present disclosure relates to the field of hydrogenation for the production of sugar alcohols, and in particular, to a method for continuous gas-liquid countercurrent preparing sugar alcohols.

### BACKGROUND

Existing sugar alcohols refer to a class of functional polyols made from biomass sugar by hydrogenation and reduction, including maltitol, sorbitol, mannitol, xylitol, lactitol, etc., which are important raw materials and products in industries such as food, fine chemicals, and pharmaceuticals. Compared with common aldoses or ketoses, biomass sugar alcohols have the advantages of low caloric value, low glycemic response, and non-cariogenic properties, making them globally recognized as safe and healthy food products.

At present, the main production process for biomass sugar alcohols is batch hydrogenation in batch reactors, where catalysts are easily lost by mechanical agitation, and the catalysts are difficult to recover. In addition, in order to ensure the thorough hydrogenation of biomass sugar in the industrial production process, it is common to continuously replenish the hydrogen gas to maintain the hydrogen gas supersaturation state in the sugar solution. To address the above problems, the continuous hydrogenation process has been progressively developed and applied in this field. U.S. Pat. No. 8,816,068B2 discloses a continuous catalytic hydrogenation process for preparing high-purity sugar alcohols, which employs two sets of fixed-bed reactors in series to achieve continuous hydrogenation of glucose. The process has an excellent glucose conversion rate and sorbitol selectivity. However, the catalysts loaded in the reactor are ruthenium-based and platinum-based catalysts, which are relatively expensive. Additionally, the process in patent U.S. Pat. No. 8,816,068B2 is characterized by a high cost, a high hydrogen consumption, and a high pressure of the hydrogen gas, limiting its industrialization. Patent WO2018118854A1 publishes a method for the preparation of maltitol by continuous hydrogenation of maltose. The method uses a fixed-bed reactor to achieve a high yield (>90%), but the technique has severe limitations in terms of operating conditions, such as the molar ratio of hydrogen gas to maltose (>40:1) and the reaction pressure of hydrogen gas (12.4 MPa-17.2 MPa). Chinese patent CN208949158U published a device for preparing sorbitol by continuous hydrogenation of glucose, combining a slurry-bed reactor connects with a fixed-bed reactor sequentially, but the total material residence time is long, the production efficiency is low, and there are cost and safety issues.

In summary, the current production process of biomass sugar alcohols still suffers from a large molar ratio of the hydrogen gas to substrate, a high consumption of the hydrogen gas, and a low production efficiency, which need to be improved. Therefore, it is desirable to provide a method for continuous gas-liquid countercurrent preparing sugar alcohol to realize the continuous hydrogenation of the biomass sugar solution by highly efficiently intensifying the gas-liquid-solid three-phase mass transfer and shortening the residence time, and substantially improve the conversion rate of the biomass sugar and the yield of the corresponding sugar alcohol.

### SUMMARY

The technical objective of the present disclosure is to provide a method for continuous gas-liquid countercurrent preparing sugar alcohol, which realizes the continuous hydrogenation of a biomass sugar solution by highly efficiently intensifying the gas-liquid-solid three-phase mass transfer and shortening the residence time, and substantially improves the conversion rate of the biomass sugar and the yield of the corresponding sugar alcohol.

One or more embodiments of the present disclosure provide a method for continuous gas-liquid countercurrent preparing sugar alcohol, wherein the method includes: through a trickle bed reactor, pouring a biomass sugar solution from a liquid phase inlet of the trickle bed reactor into the trickle bed reactor from top to bottom; and introducing hydrogen gas from a gas phase inlet of the trickle bed reactor to the trickle bed reactor from bottom to top, wherein the biomass sugar solution and the hydrogen gas pass through a catalyst bed of the trickle bed reactor in a gas-liquid countercurrent manner for hydrogenation reduction to obtain a biomass sugar alcohol solution.

### BRIEF DESCRIPTION OF THE DRAWINGS

This present disclosure will further illustrate through exemplary embodiments, which will be described in detail through the accompanying drawings. These embodiments are not limiting, and in these embodiments, the same numbering represents the same structure, where:

FIG. 1 is a schematic structural diagram of a trickle bed reactor according to some embodiments of the present disclosure.

Markings in FIG. 1 denote: 11, liquid phase inlet; 12, gas phase outlet; 13, gas phase inlet; 14, liquid phase outlet; 15, upper layer of quartz sand filler; 16, catalyst bed; 17, lower layer of quartz sand filler; 18, support frame.

FIG. 2 is a schematic structural diagram of a gas-liquid concurrent trickle bed reactor in comparative example 1 of the present disclosure.

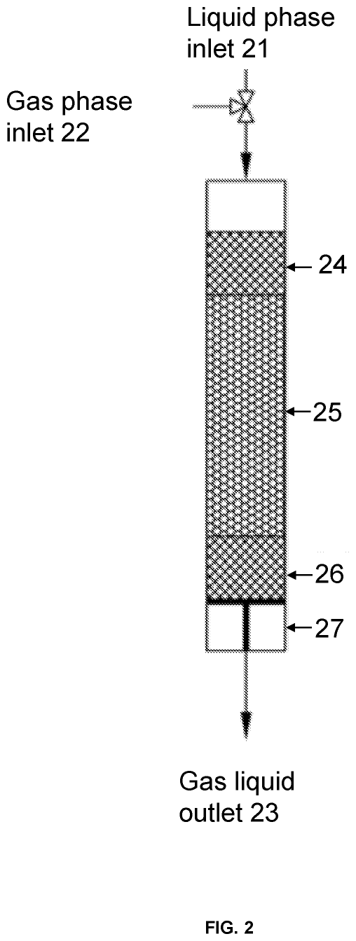

Markings in FIG. 2 denote: 21, liquid phase inlet; 22, gas phase inlet; 23, gas liquid outlet; 24, upper layer of quartz sand filler; 25, catalyst bed; 26, lower layer of quartz sand filler; 27, support frame.

### DETAILED DESCRIPTION

In order to make the above objectives, features, and advantages of the present disclosure more obvious and understandable, the following specific embodiments of the present disclosure are described in detail in conjunction with the embodiments.

Many specific details are set forth in the following description in order to facilitate a full understanding of the present disclosure, but the present disclosure may be practiced in other ways than those described herein, and a person skilled in the art may do similarly without violating the connotation of the present disclosure. Therefore, the present disclosure is not limited by the embodiments disclosed below.

Secondly, the term “one embodiment” or “embodiments” referred to herein refers to specific features, structures, or characteristics that may be included in at least one implementation of the present disclosure. The term ‘in one embodiment’ appearing in different places in this specification does not necessarily refer to the same embodiment, nor is it a separate or selective embodiment that is mutually exclusive with other embodiments.

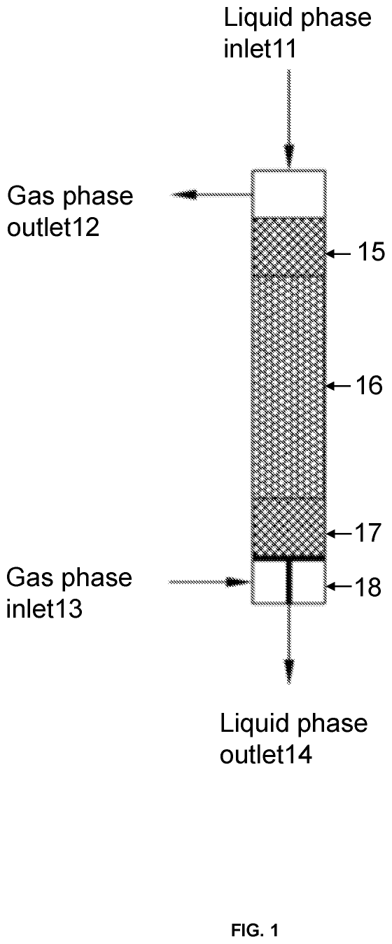

FIG. 1 is a schematic structural diagram of a trickle bed reactor according to some embodiments of the present disclosure.

In some embodiments, the method for continuous gas-liquid countercurrent preparing sugar alcohol includes: through a trickle bed reactor (also referred to as a gas-liquid countercurrent trickle bed reactor), pouring a biomass sugar solution from a liquid phase inlet of the trickle bed reactor into the trickle bed reactor from top to bottom; and introducing hydrogen gas from a gas phase inlet of the trickle bed reactor to the trickle bed reactor from bottom to top, wherein the biomass sugar solution and the hydrogen gas pass through a catalyst bed of the trickle bed reactor in a gas-liquid countercurrent manner for hydrogenation reduction to obtain a biomass sugar alcohol solution.

In some embodiments, as shown in FIG. 1, the present disclosure provides a method for continuous gas-liquid countercurrent preparing sugar alcohol: through the trickle bed reactor, a catalyst bed 16 is loaded on top of a support frame 18, and the biomass sugar solution flows into the trickle bed reactor from a liquid phase inlet 11 in a top-down direction, and the hydrogen gas flows into the trickle bed reactor from a gas phase inlet 13 a down-top direction. The biomass sugar solution and the hydrogen gas are in a gas-liquid countercurrent manner (the biomass sugar solution passes an upper layer of quartz sand filler 15, the catalyst bed 16, and a lower layer of quartz sand filler 17 in sequence, the hydrogen gas passes the lower layer of quartz sand filler 17, the catalyst bed 16, and the upper layer of quartz sand filler 15 in sequence) for hydrogenation reduction in the trickle bed reactor (e.g., the catalyst bed 16, etc.) to obtain the biomass sugar alcohol solution, and the biomass sugar alcohol solution is obtain from a liquid phase outlet 14.

The catalyst bed refers to a fixed layer formed by the filling of catalysts in the trickle bed reactor. Quartz sand filler is used to disperse the hydrogen gas and the biomass sugar solution, ensuring sufficient contact between the hydrogen gas, the biomass sugar solution, and the catalysts. The support frame is used to support the quartz sand filler and the catalyst bed.

In some embodiments, the flow direction of the biomass sugar solution from top to bottom may be a direction from the end with a higher vertical height to the end with a lower vertical height. The flow direction of the hydrogen gas from bottom to top may be a direction from the end with a lower vertical height to the end with a higher vertical height.

In some embodiments, the catalyst used in the catalyst bed 16 may include a plurality of catalysts, such as at least one of Raney nickel, Raney copper, ruthenium carbon, platinum carbon, etc. These catalysts are relatively inexpensive and have high catalytic efficiency.

In some embodiments, the particle size range of the catalyst may be predetermined. For example, the particle size of the catalyst is in a range of 1 mm-6 mm, etc. As another example, the particle size of the catalyst is in a range of 2 mm-5 mm, etc. As another example, the particle size of the catalyst is in a range of 3 mm-4 mm, etc. The particle size range may be a range in which the average particle size of catalyst particles is located.

In some embodiments of the present disclosure, the particle size range of the catalyst can effectively avoid the problem of pressure drop caused by catalyst particles that are too small, and also avoid the uneven dispersion of liquid caused by particles that are too large.

In some embodiments, the biomass sugar in the biomass sugar solution may include at least one of maltose, glucose, mannose, xylose, lactose, etc. The sugar alcohol in the biomass sugar alcohol solution may include at least one of maltitol, sorbitol, mannitol, xylitol, lactitol, etc.

In some embodiments, the types of the biomass sugar in the biomass sugar solution correspond to the types of sugar alcohol in the biomass sugar alcohol solution, for example, maltose corresponds to maltitol, glucose corresponds to sorbitol, mannose corresponds to mannitol, and xylose or lactose corresponds to xylitol or lactitol, etc.

In some embodiments, the molar concentration of the biomass sugar solution may be predetermined. For example, a molar concentration of the biomass sugar solution is in a range of 0.6 mol/L-2.2 mol/L, etc. As another example, a molar concentration of the biomass sugar solution is in a range of 1.0 mol/L-1.8 mol/L, etc. As another example, a molar concentration of the biomass sugar solution is in a range of 1.2 mol/L-1.6 mol/L, etc.

In some embodiments of the present disclosure, the molar concentration of the biomass sugar solution can effectively enhance the product concentration and reduce the cost of subsequent crystallization and separation on the basis of ensuring the conversion efficiency.

In some embodiments, the biomass sugar solution is continuously fed using a liquid-phase pump or other devices through the liquid phase inlet 11, and the feed flow rate range of the biomass sugar solution may be predetermined. For example, the feed flow rate of the biomass sugar solution is in a range of 0.5 mL/min-5 mL/min, etc. As another example, the feed flow rate of the biomass sugar solution is in a range of 1 mL/min-4 mL/min, etc. As another example, the feed flow rate of the biomass sugar solution is in a range of 2 mL/min-3 mL/min, etc.

In some embodiments of the present disclosure, the feed flow rate range is conducive to prolonging the residence time of the liquid on the surface of the catalyst, enhancing the yield of the sugar alcohol, and effectively preventing the hydrolysis side reaction caused by local overheating.

In some embodiments, the gas pressure range of the hydrogen gas may be predetermined. For example, the gas pressure of the hydrogen gas is in a range of 5-10 MPa, etc. As another example, the gas pressure of the hydrogen gas is in a range of 6-9 MPa, etc. As another example, the gas pressure of the hydrogen gas is in a range of 7-8 MPa, etc.

In some embodiments of the present disclosure, the gas pressure range of the hydrogen gas can ensure the excellent conversion rate of the biomass sugar and the yield of the sugar alcohol, while taking into account safety and economic considerations.

In some embodiments, the gas flow rate of the hydrogen gas is controlled using a mass flow meter, etc. The gas flow rate of the hydrogen gas may be predetermined. For example, the flow rate of the hydrogen gas is in a range of 10-100 sccm, etc. As another example, the flow rate of the hydrogen gas is in a range of 30-70 sccm, etc. As another example, the flow rate of the hydrogen gas is in a range of 50-60 sccm, etc.

In some embodiments, the reaction temperature range of the trickle bed reactor may be predetermined. For example, the reaction temperature of the trickle bed reactor is in a range of 100-140° C. As another example, the reaction temperature of the trickle bed reactor is in a range of 110-130° C. As another example, the reaction temperature of the trickle bed reactor is in a range of 115-125° C.

In some embodiments of the present disclosure, the reaction temperature range of the trickle bed reactor can balance high conversion and high selectivity to maximize the generation of target sugar alcohols.

In some embodiments, a feed flow rate of the biomass sugar solution is in a range of 1 mL/min-2 mL/min, the gas pressure of the hydrogen gas is in a of 6 MPa-8 MPa, and the hydrogen gas is at a gas flow rate range of 20-80 sccm, and a reaction temperature range of the trickle bed reactor is 120-135° C.

In some embodiments, the particle size of the catalyst is in a range of 1 mm-4 mm, the molar concentration of the biomass sugar solution is in a range of 0.6 mol/L-1.8 mol/L, the feed flow rate of the biomass sugar solution is in a range of 1 mL/min-2 mL/min, the gas pressure of the hydrogen gas is in a range of 6 Mpa-9 MPa, the flow rate of the hydrogen gas is in a range of 20 sccm-80 sccm, and the reaction temperature of the trickle bed reactor is in a range of 120° C.-140° C. The above-described process conditions can achieve a conversion rate of the biomass sugar of at least 87% and the yield of the sugar alcohol of at least 83%.

In some embodiments, the conversion rate of the biomass sugar may be expressed as a ratio of the amount of the biomass sugar consumed to the total amount of biomass sugar entering the trickle bed reactor. The yield of sugar alcohol may be expressed as a ratio of the amount of biomass sugar alcohol produced to the theoretical maximum yield of biomass sugar alcohol.

In some embodiments, the particle size of the catalyst is in a range of 1 mm-3.5 mm, the molar concentration of the biomass sugar solution is in a range of 0.6 mol/L-1.6 mol/L, the feed flow rate of the biomass sugar solution is in a range of 1 mL/min-1.5 mL/min, the pressure of the hydrogen gas is in a range of 6 Mpa-9 MPa, the flow rate of the hydrogen gas is in a range of 20 sccm-70 sccm, and the reaction temperature of the trickle bed reactor is in a range of 120° C.-140° C. The above-described process conditions can achieve the conversion rate of the biomass sugar of at least 92% and the yield of the sugar alcohol of at least 90%.

In some embodiments, the particle size of the catalyst is in a range of 1 mm-3.5 mm, the molar concentration of the biomass sugar solution is in a range of 1 mol/L-1.6 mol/L, the feed flow rate of the biomass sugar solution is in a range of 1 mL/min-1.5 mL/min, the pressure of the hydrogen gas is in a range of 6 Mpa-9 MPa, the flow rate of the hydrogen gas is in a range of 20 sccm-50 sccm, and the reaction temperature of the trickle bed reactor is in a range of 120° C.-140° C. The above-described process conditions can achieve the conversion rate of the biomass sugar of at least 97.5% and the yield of the sugar alcohol of at least 94.5%.

In some embodiments, the particle size of the catalyst is in a range of 2 mm-3 mm, the molar concentration of the biomass sugar solution is in a range of 1.4 mol/L-1.6 mol/L, the feed flow rate of the biomass sugar solution is 1 mL/min, the pressure of the hydrogen gas is in a range of 6 Mpa-8 MPa, the flow rate of the hydrogen gas is in a range of 20 sccm-50 sccm, and the reaction temperature of the trickle bed reactor is in a range of 120° C.-130° C. The above-described process conditions can achieve the conversion rate of the biomass sugar of at least 99% and the yield of the sugar alcohol of at least 98%.

In some embodiments of the present disclosure, compared with the existing continuous hydrogenation process of biomass sugar, the present disclosure adopts a gas-liquid countercurrent trickle bed reactor to prepare the corresponding sugar alcohol, which can effectively ensure the full contact among the hydrogen gas, the biomass sugar solution, and the catalyst. By strengthening the three-phase mass transfer, the residence time of the material is shortened, thereby improving the production efficiency of sugar alcohol.

In some embodiments of the present disclosure, the pressure and the flow rate of the hydrogen gas are lower, and the amount of hydrogen gas is smaller, which greatly reduces the production cost and effectively avoids the safety hazards caused by high-pressure hydrogen gas.

Some embodiments of the present disclosure are applicable to the continuous hydrogenation of a plurality of biomass sugars, and can maintain a good conversion rate of the biomass sugar and a good yield of the sugar alcohol (including space-time yield) under high concentration material conditions (such as 1.2-1.8 mol/L), with excellent industrialization and scaling-up potential.

Some embodiments of the present disclosure can be widely used in the industrialized preparation of functional sugar alcohols such as maltitol, sorbitol, mannitol, xylitol, lactitol, or the like.

### Example 1

The gas-liquid countercurrent trickle bed reactor was filled with 90 g of Raney nickel catalyst (with an average particle size of 1 mm), the trickle bed reactor was heated up to 130° C., the air inside the trickle bed reactor was replaced and expelled with high-purity nitrogen (e.g., nitrogen gas with a total impurity content of ≤10 ppm, etc.), and then 30 mL/min of hydrogen gas was introduced into the trickle bed reactor from the gas phase inlet 13 at the lower end of the trickle bed reactor and flowed out from a gas phase outlet 12 at the upper end of the trickle bed reactor, and the pressure inside the trickle bed reactor was controlled by a back-pressure valve at 8 MPa. Meanwhile, a liquid-phase metering pump or other device was used to add 1.4 mol/L maltose solution to the trickle bed reactor from the liquid phase inlet 11 at the top end of the trickle bed reactor at a flow rate of 1 mL/min, so that the hydrogen gas and the maltose solution can pass through the catalyst bed 16 in the gas-liquid countercurrent manner for continuous hydrogenation reaction, and the maltose hydrogenation solution is obtained from the liquid phase outlet 14 at the bottom of the trickle bed reactor.

The sample of the above maltose hydrogenation solution in Example 1 was analyzed by high-performance liquid chromatography (HPLC) or other manner, and the results include that: a conversion rate of the maltose is 97.5%, and a yield of the maltitol is 94.8%.

### Example 2

The gas-liquid countercurrent trickle bed reactor was filled with 90 g of Raney nickel catalyst (with an average particle size of 3 mm), the trickle bed reactor was heated up to 130° C., the air inside the trickle bed reactor was replaced and expelled with high-purity nitrogen, and then 30 mL/min of hydrogen gas was introduced into the trickle bed reactor from the gas phase inlet 13 at the lower end of the trickle bed reactor and flowed out from the gas phase outlet 12 at the upper end of the trickle bed reactor, and the pressure inside the trickle bed reactor was controlled by the back-pressure valve at 8 MPa. Meanwhile, the liquid-phase metering pump or other device was used to add 1.4 mol/L maltose solution to the trickle bed reactor from the liquid phase inlet 11 at the top end of the trickle bed reactor at a flow rate of 3 mL/min, so that the hydrogen gas and the maltose solution can pass through the catalyst bed 16 in the gas-liquid countercurrent manner for continuous hydrogenation reaction, and the maltose hydrogenation solution is obtained from the liquid phase outlet 14 at the bottom of the trickle bed reactor.

The sample of the above maltose hydrogenation solution in Example 2 was analyzed by high-performance liquid chromatography (HPLC) or other manner, and the results include that: a conversion rate of the maltose is 70.4%, and a yield of the maltitol is 67.9%.

### Example 3

The gas-liquid countercurrent trickle bed reactor was filled with 90 g of Raney nickel catalyst (with an average particle size of 4 mm), the trickle bed reactor was heated up to 130° C., the air inside the trickle bed reactor was replaced and expelled with high-purity nitrogen, and then 30 mL/min of hydrogen gas was introduced into the trickle bed reactor from the gas phase inlet 13 at the lower end of the trickle bed reactor and flowed out from the gas phase outlet 12 at the upper end of the trickle bed reactor, and the pressure inside the trickle bed reactor was controlled by the back-pressure valve at 8 MPa. Meanwhile, the liquid-phase metering pump or other device was used to add 1.4 mol/L maltose solution to the trickle bed reactor from the liquid phase inlet 11 at the top end of the trickle bed reactor at a flow rate of 5 mL/min, so that the hydrogen gas and the maltose solution can pass through the catalyst bed 16 in the gas-liquid countercurrent manner for continuous hydrogenation reaction, and the maltose hydrogenation solution is obtained from the liquid phase outlet 14 at the bottom of the trickle bed reactor.

The sample of the above maltose hydrogenation solution in Example 3 was analyzed by high-performance liquid chromatography (HPLC) or other manner, and the results include that: a conversion rate of the maltose is 55.1%, and a yield of the maltitol is 53.5%.

### Example 4

The gas-liquid countercurrent trickle bed reactor was filled with 80 g of Raney nickel catalyst (with an average particle size of 3 mm), the trickle bed reactor was heated up to 110° C., the air inside the trickle bed reactor was replaced and expelled with high-purity nitrogen, and then 50 mL/min of hydrogen gas was introduced into the trickle bed reactor from the gas phase inlet 13 at the lower end of the trickle bed reactor and flowed out from the gas phase outlet 12 at the upper end of the trickle bed reactor, and the pressure inside the trickle bed reactor was controlled by the back-pressure valve at 7.5 MPa. Meanwhile, the liquid-phase metering pump or other device was used to add 1.4 mol/L maltose solution to the trickle bed reactor from the liquid phase inlet 11 at the top end of the trickle bed reactor at a flow rate of 1.5 mL/min, so that the hydrogen gas and the maltose solution can pass through the catalyst bed 16 in the gas-liquid countercurrent manner for continuous hydrogenation reaction, and the maltose hydrogenation solution is obtained from the liquid phase outlet 14 at the bottom of the trickle bed reactor.

The sample of the above maltose hydrogenation solution in Example 4 was analyzed by high-performance liquid chromatography (HPLC) or other manner, and the results include that: a conversion rate of the maltose is 75.5%, and a yield of the maltitol is 69.3%.

### Example 5

The gas-liquid countercurrent trickle bed reactor was filled with 80 g of Raney nickel catalyst (with an average particle size of 3 mm), the trickle bed reactor was heated up to 120° C., the air inside the trickle bed reactor was replaced and expelled with high-purity nitrogen, and then 50 mL/min of hydrogen gas was introduced into the trickle bed reactor from the gas phase inlet 13 at the lower end of the trickle bed reactor and flowed out from the gas phase outlet 12 at the upper end of the trickle bed reactor, and the pressure inside the trickle bed reactor was controlled by the back-pressure valve at 7.5 MPa. Meanwhile, the liquid-phase metering pump or other device was used to add 1.4 mol/L maltose solution to the trickle bed reactor from the liquid phase inlet 11 at the top end of the trickle bed reactor at a flow rate of 1.5 mL/min, so that the hydrogen gas and the maltose solution can pass through the catalyst bed 16 in the gas-liquid countercurrent manner for continuous hydrogenation reaction, and the maltose hydrogenation solution is obtained from the liquid phase outlet 14 at the bottom of the trickle bed reactor.

The sample of the above maltose hydrogenation solution in Example 5 was analyzed by high-performance liquid chromatography (HPLC) or other manner, and the results include that: a conversion rate of the maltose is 87.2%, and a yield of the maltitol is 83.3%.

### Example 6

The gas-liquid countercurrent trickle bed reactor was filled with 80 g of Raney nickel catalyst (with an average particle size of 3 mm), the trickle bed reactor was heated up to 140° C., the air inside the trickle bed reactor was replaced and expelled with high-purity nitrogen, and then 50 mL/min of hydrogen gas was introduced into the trickle bed reactor from the gas phase inlet 13 at the lower end of the trickle bed reactor and flowed out from the gas phase outlet 12 at the upper end of the trickle bed reactor, and the pressure inside the trickle bed reactor was controlled by the back-pressure valve at 7.5 MPa. Meanwhile, the liquid-phase metering pump or other device was used to add 1.4 mol/L maltose solution to the trickle bed reactor from the liquid phase inlet 11 at the top end of the trickle bed reactor at a flow rate of 1.5 mL/min, so that the hydrogen gas and the maltose solution can pass through the catalyst bed 16 in the gas-liquid countercurrent manner for continuous hydrogenation reaction, and the maltose hydrogenation solution is obtained from the liquid phase outlet 14 at the bottom of the trickle bed reactor.

The sample of the above maltose hydrogenation solution in Example 6 was analyzed by high-performance liquid chromatography (HPLC) or other manner, and the results include that: a conversion rate of the maltose is 98.3%, and a yield of the maltitol is 94.5%.

### Example 7

The gas-liquid countercurrent trickle bed reactor was filled with 100 g of Raney nickel catalyst (with an average particle size of 3 mm), the trickle bed reactor was heated up to 125° C., the air inside the trickle bed reactor was replaced and expelled with high-purity nitrogen, and then 70 mL/min of hydrogen gas was introduced into the trickle bed reactor from the gas phase inlet 13 at the lower end of the trickle bed reactor and flowed out from the gas phase outlet 12 at the upper end of the trickle bed reactor, and the pressure inside the trickle bed reactor was controlled by the back-pressure valve at 6 MPa. Meanwhile, the liquid-phase metering pump or other device was used to add 1.4 mol/L maltose solution to the trickle bed reactor from the liquid phase inlet 11 at the top end of the trickle bed reactor at a flow rate of 1 mL/min, so that the hydrogen gas and the maltose solution can pass through the catalyst bed 16 in the gas-liquid countercurrent manner for continuous hydrogenation reaction, and the maltose hydrogenation solution is obtained from the liquid phase outlet 14 at the bottom of the trickle bed reactor.

The sample of the above maltose hydrogenation solution in Example 7 was analyzed by high-performance liquid chromatography (HPLC) or other manner, and the results include that: a conversion rate of the maltose is 87.6%, and a yield of the maltitol is 84.4%.

### Example 8

The gas-liquid countercurrent trickle bed reactor was filled with 100 g of Raney nickel catalyst (with an average particle size of 3 mm), the trickle bed reactor was heated up to 125° C., the air inside the trickle bed reactor was replaced and expelled with high-purity nitrogen, and then 70 mL/min of hydrogen gas was introduced into the trickle bed reactor from the gas phase inlet 13 at the lower end of the trickle bed reactor and flowed out from the gas phase outlet 12 at the upper end of the trickle bed reactor, and the pressure inside the trickle bed reactor was controlled by the back-pressure valve at 7 MPa. Meanwhile, the liquid-phase metering pump or other device was used to add 1.4 mol/L maltose solution to the trickle bed reactor from the liquid phase inlet 11 at the top end of the trickle bed reactor at a flow rate of 1 mL/min, so that the hydrogen gas and the maltose solution can pass through the catalyst bed 16 in the gas-liquid countercurrent manner for continuous hydrogenation reaction, and the maltose hydrogenation solution is obtained from the liquid phase outlet 14 at the bottom of the trickle bed reactor.

The sample of the above maltose hydrogenation solution in Example 8 was analyzed by high-performance liquid chromatography (HPLC) or other manner, and the results include that: a conversion rate of the maltose is 92.3%, and a yield of the maltitol is 90.0%.

### Example 9

The gas-liquid countercurrent trickle bed reactor was filled with 100 g of Raney nickel catalyst (with an average particle size of 2.5 mm), the trickle bed reactor was heated up to 130° C., the air inside the trickle bed reactor was replaced and expelled with high-purity nitrogen, and then 30 mL/min of hydrogen gas was introduced into the trickle bed reactor from the gas phase inlet 13 at the lower end of the trickle bed reactor and flowed out from the gas phase outlet 12 at the upper end of the trickle bed reactor, and the pressure inside the trickle bed reactor was controlled by the back-pressure valve at 9 MPa. Meanwhile, the liquid-phase metering pump or other device was used to add 0.6 mol/L maltose solution to the trickle bed reactor from the liquid phase inlet 11 at the top end of the trickle bed reactor at a flow rate of 1 mL/min, so that the hydrogen gas and the maltose solution can pass through the catalyst bed 16 in the gas-liquid countercurrent manner for continuous hydrogenation reaction, and the maltose hydrogenation solution is obtained from the liquid phase outlet 14 at the bottom of the trickle bed reactor.

The sample of the above maltose hydrogenation solution in Example 9 was analyzed by high-performance liquid chromatography (HPLC) or other manner, and the results include that: a conversion rate of the maltose is 99.4%, and a yield of the maltitol is 96.9%.

### Example 10

The gas-liquid countercurrent trickle bed reactor was filled with 100 g of Raney nickel catalyst (with an average particle size of 2.5 mm), the trickle bed reactor was heated up to 130° C., the air inside the trickle bed reactor was replaced and expelled with high-purity nitrogen, and then 30 mL/min of hydrogen gas was introduced into the trickle bed reactor from the gas phase inlet 13 at the lower end of the trickle bed reactor and flowed out from the gas phase outlet 12 at the upper end of the trickle bed reactor, and the pressure inside the trickle bed reactor was controlled by the back-pressure valve at 9 MPa. Meanwhile, the liquid-phase metering pump or other device was used to add 1.0 mol/L maltose solution to the trickle bed reactor from the liquid phase inlet 11 at the top end of the trickle bed reactor at a flow rate of 1 mL/min, so that the hydrogen gas and the maltose solution can pass through the catalyst bed 16 in the gas-liquid countercurrent manner for continuous hydrogenation reaction, and the maltose hydrogenation solution is obtained from the liquid phase outlet 14 at the bottom of the trickle bed reactor.

The sample of the above maltose hydrogenation solution in Example 10 was analyzed by high-performance liquid chromatography (HPLC) or other manner, and the results include that: a conversion rate of the maltose is 98.7%, and a yield of the maltitol is 95.3%.

### Example 11

The gas-liquid countercurrent trickle bed reactor was filled with 100 g of Raney nickel catalyst (with an average particle size of 2.5 mm), the trickle bed reactor was heated up to 130° C., the air inside the trickle bed reactor was replaced and expelled with high-purity nitrogen, and then 30 mL/min of hydrogen gas was introduced into the trickle bed reactor from the gas phase inlet 13 at the lower end of the trickle bed reactor and flowed out from the gas phase outlet 12 at the upper end of the trickle bed reactor, and the pressure inside the trickle bed reactor was controlled by the back-pressure valve at 9 MPa. Meanwhile, the liquid-phase metering pump or other device was used to add 2 mol/L maltose solution to the trickle bed reactor from the liquid phase inlet 11 at the top end of the trickle bed reactor at a flow rate of 1 mL/min, so that the hydrogen gas and the maltose solution can pass through the catalyst bed 16 in the gas-liquid countercurrent manner for continuous hydrogenation reaction, and the maltose hydrogenation solution is obtained from the liquid phase outlet 14 at the bottom of the trickle bed reactor.

The sample of the above maltose hydrogenation solution in Example 11 was analyzed by high-performance liquid chromatography (HPLC) or other manner, and the results include that: a conversion rate of the maltose is 88.2%, and a yield of the maltitol is 86.1%.

### Example 12

The gas-liquid countercurrent trickle bed reactor was filled with 100 g of Raney nickel catalyst (with an average particle size of 3 mm), the trickle bed reactor was heated up to 130° C., the air inside the trickle bed reactor was replaced and expelled with high-purity nitrogen, and then 50 mL/min of hydrogen gas was introduced into the trickle bed reactor from the gas phase inlet 13 at the lower end of the trickle bed reactor and flowed out from the gas phase outlet 12 at the upper end of the trickle bed reactor, and the pressure inside the trickle bed reactor was controlled by the back-pressure valve at 7 MPa. Meanwhile, the liquid-phase metering pump or other device was used to add 1.6 mol/L glucose solution to the trickle bed reactor from the liquid phase inlet 11 at the top end of the trickle bed reactor at a flow rate of 1 mL/min, so that the hydrogen gas and the glucose solution can pass through the catalyst bed 16 in the gas-liquid countercurrent manner for continuous hydrogenation reaction, and the glucose hydrogenation solution is obtained from the liquid phase outlet 14 at the bottom of the trickle bed reactor.

The sample of the above glucose hydrogenation solution in Example 12 was analyzed by high-performance liquid chromatography (HPLC) or other manner, and the results include that: a conversion rate of the glucose is 99.1%, and a yield of the sorbitol is 98.0%.

### Example 13

The gas-liquid countercurrent trickle bed reactor was filled with 90 g of Raney nickel catalyst (with an average particle size of 2 mm), the trickle bed reactor was heated up to 125° C., the air inside the trickle bed reactor was replaced and expelled with high-purity nitrogen, and then 20 mL/min of hydrogen gas was introduced into the trickle bed reactor from the gas phase inlet 13 at the lower end of the trickle bed reactor and flowed out from the gas phase outlet 12 at the upper end of the trickle bed reactor, and the pressure inside the trickle bed reactor was controlled by the back-pressure valve at 8 MPa. Meanwhile, the liquid-phase metering pump or other device was used to add 1.4 mol/L mannose solution to the trickle bed reactor from the liquid phase inlet 11 at the top end of the trickle bed reactor at a flow rate of 1 mL/min, so that the hydrogen gas and the mannose solution can pass through the catalyst bed 16 in the gas-liquid countercurrent manner for continuous hydrogenation reaction, and the mannose hydrogenation solution is obtained from the liquid phase outlet 14 at the bottom of the trickle bed reactor.

The sample of the above mannose hydrogenation solution in Example 13 was analyzed by high-performance liquid chromatography (HPLC) or other manner, and the results include that: a conversion rate of the mannose is 99.7%, and a yield of the mannitol is 98.5%.

### Example 14

The gas-liquid countercurrent trickle bed reactor was filled with 80 g of Raney nickel catalyst (with an average particle size of 2.5 mm), the trickle bed reactor was heated up to 120° C., the air inside the trickle bed reactor was replaced and expelled with high-purity nitrogen, and then 30 mL/min of hydrogen gas was introduced into the trickle bed reactor from the gas phase inlet 13 at the lower end of the trickle bed reactor and flowed out from the gas phase outlet 12 at the upper end of the trickle bed reactor, and the pressure inside the trickle bed reactor was controlled by the back-pressure valve at 6 MPa. Meanwhile, the liquid-phase metering pump or other device was used to add 1.4 mol/L xylose solution to the trickle bed reactor from the liquid phase inlet 11 at the top end of the trickle bed reactor at a flow rate of 1 mL/min, so that the hydrogen gas and the xylose solution can pass through the catalyst bed 16 in the gas-liquid countercurrent manner for continuous hydrogenation reaction, and the xylose hydrogenation solution is obtained from the liquid phase outlet 14 at the bottom of the trickle bed reactor.

The sample of the above xylose hydrogenation solution in Example 14 was analyzed by high-performance liquid chromatography (HPLC) or other manner, and the results include that: a conversion rate of the xylose is 99.9%, and a yield of the xylitol is 98.7%.

### Example 15

The gas-liquid countercurrent trickle bed reactor was filled with 80 g of Raney nickel catalyst (with an average particle size of 3.5 mm), the trickle bed reactor was heated up to 130° C., the air inside the trickle bed reactor was replaced and expelled with high-purity nitrogen, and then 40 mL/min of hydrogen gas was introduced into the trickle bed reactor from the gas phase inlet 13 at the lower end of the trickle bed reactor and flowed out from the gas phase outlet 12 at the upper end of the trickle bed reactor, and the pressure inside the trickle bed reactor was controlled by the back-pressure valve at 8 MPa. Meanwhile, the liquid-phase metering pump or other device was used to add 1.2 mol/L lactose solution to the trickle bed reactor from the liquid phase inlet 11 at the top end of the trickle bed reactor at a flow rate of 1 mL/min, so that the hydrogen gas and the lactose solution can pass through the catalyst bed 16 in the gas-liquid countercurrent manner for continuous hydrogenation reaction, and the lactose hydrogenation solution is obtained from the liquid phase outlet 14 at the bottom of the trickle bed reactor.

The sample of the above lactose hydrogenation solution in Example 15 was analyzed by high-performance liquid chromatography (HPLC) or other manner, and the results include that: a conversion rate of the lactose is 97.3%, and a yield of the lactitol is 95.5%.

### Comparative Example 1

FIG. 2 is a schematic structural diagram of a gas-liquid concurrent trickle bed reactor in comparative example 1 of the present disclosure.

As shown in FIG. 2, a gas-liquid concurrent trickle bed reactor was filled with 80 g of Raney nickel catalyst (with an average particle size of 3 mm) above a support frame 27, the trickle bed reactor was heated up to 130° C., the air inside the trickle bed reactor was replaced and expelled with high-purity nitrogen, and then 30 mL/min of hydrogen gas was introduced into the trickle bed reactor from a gas phase inlet 22 at the lower end of the trickle bed reactor, and the pressure inside the trickle bed reactor was controlled by the back-pressure valve at 8 MPa. Meanwhile, the liquid-phase metering pump or other device was used to add 1.4 mol/L maltose solution to the trickle bed reactor from a liquid phase inlet 21 at the top end of the trickle bed reactor at a flow rate of 1 mL/min, so that after being mixed by a T-tube, the hydrogen gas and maltose solution can pass through an upper layer of quartz sand filler 24, a catalyst bed 25, and a lower layer of quartz sand filler 26 in succession for continuous hydrogenation reaction, and the maltose hydrogenation solution is obtained from a gas-liquid outlet 23 at the bottom of the trickle bed reactor after the gas-liquid separation.

The sample of the above maltose hydrogenation solution in Comparative example 1 was analyzed by high-performance liquid chromatography (HPLC) or other manner, and the results include that: a conversion rate of the maltose is 73.1%, and a yield of the maltitol is 69.7%.

As can be seen from Example 1, Examples 5-11 and Comparative Example 1, under similar conditions, compared to the process in which the maltose solution and the hydrogen are continuously reacted by hydrogenation in a gas-liquid concurrent manner, the conversion rate of the maltose and the yield of the maltitol corresponding to the process in which the maltose solution and the hydrogen are continuously reacted by hydrogenation in the gas-liquid countercurrent manner are significantly improved.

In some embodiments, the method for continuous gas-liquid countercurrent preparing sugar alcohol, wherein the method includes: in a gas-liquid countercurrent trickle bed reactor, pouring a biomass sugar solution from a liquid phase inlet into the trickle bed reactor from top to bottom; and introducing hydrogen gas from a gas phase inlet to the trickle bed reactor from bottom to top, wherein the biomass sugar solution and the hydrogen gas pass through a catalyst bed in a gas-liquid countercurrent manner for hydrogenation reduction to obtain a biomass sugar alcohol solution.

In some embodiments, a catalyst includes Raney nickel, Raney copper, ruthenium carbon, or platinum carbon.

In some embodiments, an average particle size of the catalyst is in a range of 1 mm-6 mm.

In some embodiments, biomass sugar in the biomass sugar solution includes maltose, glucose, mannose, xylose, or lactose, and the sugar alcohol includes maltitol, sorbitol, mannitol, xylitol, or lactitol.

In some embodiments, a molar concentration of the biomass sugar solution is in a range of 0.6 mol/L-2.2 mol/L, and a feed flow rate of the biomass sugar solution is in a range of 0.5 mL/min-5 mL/min.

In some embodiments, a pressure of the hydrogen gas is in a range of 5 Mpa-10 MPa, and a flow rate of the hydrogen gas is in a range of 10 sccm-100 sccm.

In some embodiments, wherein a temperature of the trickle bed reactor is in a range of 100° C.-140° C.

In some embodiments, a feed flow rate of the biomass sugar solution is in a range of 1 mL/min-2 mL/min, a pressure of the hydrogen gas is in a range of 6 Mpa-8 MPa, a flow rate of the hydrogen gas is in a range of 20 sccm-80 sccm, and a temperature of the trickle bed reactor is in a range of 120° C.-135° C.

The above-described specific embodiments provide a detailed description of the technical solutions and beneficial effects of the present disclosure. It should be understood that the above description is only the most preferred embodiments of the present disclosure, and is not intended to limit the present disclosure. Any modifications, supplements, equivalent replacements, etc., made within the scope of the principles of the present disclosure shall be included in the scope of protection of the present disclosure.

What is claimed is:

1. A method for continuous gas-liquid countercurrent preparing sugar alcohol, wherein the method comprises:
through a trickle bed reactor, pouring a biomass sugar solution from a liquid phase inlet of the trickle bed reactor into the trickle bed reactor from top to bottom; and
introducing hydrogen gas from a gas phase inlet of the trickle bed reactor to the trickle bed reactor from bottom to top, wherein the biomass sugar solution and the hydrogen gas pass through a catalyst bed of the trickle bed reactor in a gas-liquid countercurrent manner for hydrogenation reduction to obtain a biomass sugar alcohol solution.

2. The method of claim 1, wherein a catalyst used in the catalyst bed includes Raney nickel, Raney copper, ruthenium carbon, or platinum carbon.

3. The method of claim 2, wherein a particle size of the catalyst is in a range of 1 mm-6 mm.

4. The method of claim 1, wherein biomass sugar in the biomass sugar solution includes at least one of maltose, glucose, mannose, xylose, or lactose, and the sugar alcohol in the biomass sugar alcohol solution includes at least one of maltitol, sorbitol, mannitol, xylitol, or lactitol.

5. The method of claim 1, wherein a molar concentration of the biomass sugar solution is in a range of 0.6 mol/L-2.2 mol/L, and a feed flow rate of the biomass sugar solution is in a range of 0.5 mL/min-5 mL/min.

6. The method of claim 1, wherein a pressure of the hydrogen gas is in a range of 5 Mpa-10 MPa, and a flow rate of the hydrogen gas is in a range of 10 sccm-100 sccm.

7. The method of claim 1, wherein a reaction temperature of the trickle bed reactor is in a range of 100° C.-140° C.

8. The method of claim 1, wherein a feed flow rate of the biomass sugar solution is in a range of 1 mL/min-2 mL/min, a pressure of the hydrogen gas is in a range of 6 Mpa-8 MPa, a flow rate of the hydrogen gas is in a range of 20 sccm-80 sccm, and a reaction temperature of the trickle bed reactor is in a range of 120° C.-135° C.
